(19) United States
(12) Patent Application Publication
(10) Pub. No.: US 2024/0024371 A1
(43) Pub. Date: Jan. 25, 2024

(54) TREATING DIABETES

(71) Applicant: SDF BioPharma Inc., Rancho Santa Fe, CA (US)

(72) Inventors: Gerald F. SWISS, Rancho Santa Fe, CA (US); David KIEWLICH, Rancho Santa Fe, CA (US)

(21) Appl. No.: 18/255,570

(22) PCT Filed: Dec. 3, 2021
(86) PCT No.: PCT/US2021/061872

§ 371 Date: Jun. 1, 2023

Related U.S. Application Data
(60) Provisional application No. 63/121,437, filed on Dec. 4, 2020.

(51) Int. Cl. A61K 35/39; A61K 9/06; A61K 38/19; A61K 38/20; A61K 38/16; A61P 3/10

(52) U.S. Cl. CPC A61K 35/39; A61K 9/06; A61K 38/195; A61K 38/2053; A61K 38/162; A61P 3/10

(57) ABSTRACT

Described herein are compositions and methods for treating a subject having diabetes comprising administering transgenic beta cells expressing fugetactic levels of CXCL12 to a subject in need thereof. Genetically engineered immune-protected, human beta cells are also described. The disclosed engineered beta cells can be administered to the subject alone or implanted into or with a biocompatible and biodegradable matrix that elutes an effective amount of a fugetactic agent, which is then implanted into the subject.

### CROSS-REFERENCE TO RELATED APPLICATIONS

This application claims the benefit under 35 U.S.C. § 119 of U.S. Provisional Application Ser. No. 63/121,437 filed on Dec. 4, 2020, which application is incorporated herein by reference in its entirety.

### FIELD OF THE INVENTION

This invention is directed to implantation methods for treating diabetes in a patient with a composition comprising a population of immune protected, human beta cells. In one embodiment, the human beta cells express an effective amount of a fugetactic agent (e.g., CXCL12) to provide protection against rejection by the patient's immune system after implantation. In one embodiment, these human beta cells are implanted into or with a rapidly biocompatible and biodegradable matrix that elutes an effective amount of a fugetactic agent so as to facilitate the survival of the implanted cells.

### REFERENCE TO A SEQUENCE LISTING

The instant application contains a Sequence Listing, which has been submitted electronically in ASCII format and is hereby incorporated by reference in its entirety. Said ASCII copy, created on Dec. 3, 2021, is named 220710_0006_WO_SL.txt and is 8,470 bytes in size.

### BACKGROUND

Beta cells are a subset of islet cells that are responsible for producing insulin in the pancreas. In patients with Type 1 Diabetes (T1D), beta cells are attacked and destroyed by their immune system, and, as a result, patients with T1D cannot efficiently produce their own insulin. As such, patients with T1D are required to closely monitor their blood glucose levels and to administer insulin as needed to maintain control over their blood glucose.

Type 2 diabetes (T2D) occurs when a subject's persistently high blood sugar overwhelms the capacity of a subject's beta cells to produce enough insulin to prevent hyperglycemia in the subject. While there are now many options for treating T2D, one study reviewing data from 1997 to 2011 reported that insulin is used, either alone or in combination with other drugs, to treat T2D in about 30% or more of all T2D patients. www.cdc.gov/diabetes/statistics/meduse/fig2.htm

One method for treating T1D is the implantation of allogeneic islet cells from a donor to a patient. However, when so treated, the patient must be on immune suppression agents to prevent infiltration of mononuclear cells (T-cells, B-cells, and NK cells) that will kill these cells. Cloning technique used to create pancreatic cells for type 1 diabetes, Diabetes.Co.Uk, www.diabetes.co.uk/news/2014/apr/cloning-technique-used-to-create-pancreatic-cells-for-type-1-diabetes-94233303.html (Apr. 29, 2014); Alan H. Cruickshank & Emyr W Benbow, “Recurrence of Diabetes,” Pathology of the Pancreas (2d ed. 1995); Felicia W Pagliuca & Douglas A. Melton, “How to make a functional β-cell,” Development 2013, 140(12), 2472-2483. Even then, a significant number of beta cells fail to survive the implantation process.

Alternatively, allogeneic and xenogeneic beta cells have been proposed for use with matrices such as alginates wherein these cells are encapsulated in the matrix and are protected against immune rejection. In one embodiment, the matrix is impregnated with fugetactic agent that elutes a sufficient amount over an extended period of time so as to provide immune protection to the encapsulated cells.

There are several problems that arise with such implantable matrices. Specifically, it is reported that implanted beta cells have a low survival rate that improves somewhat when encapsulated in macro-devices. The latter is not surprising as oxygen and nutrient availability may be compromised by encapsulation. Even assuming adequate oxygen and nutrient availability, the ability of the matrix to continuously elute an effective amount of a fugetactic agent over an extended period of time so as to provide immune protection to these beta cells is challenging at best and limited at worse. More likely, the elution of the fugetactic agent from the implant will have an effective life span that will likely vary from patient to patient.

When the useful life of the matrix is exhausted, the patient will require surgery to effect removal of the old matrix coupled with implantation of a new matrix. Any such periodic surgeries are undesirable as there is a risk of adverse events to the patient coupled with the inconvenience, pain and other attributes associated with surgery.

Genetically modified beta cells that express an effective amount of a fugetactic agent so that the cells themselves are masked, or stealth to the immune system, have been proposed. See, e.g., WO 2019/0100729, which application is incorporated herein by reference in its entirety. The so expressed agent maintains an immune repellent microenvironment around the cells that protects these cells from destruction by immune cells. This poses a problem in the direct implantation of these cells as the immune repellent microenvironment generated in vitro is not maintained when these cells are implanted in vivo. As during the time required to generate such an immune repellent microenvironment in vivo, these newly implanted beta cells are at risk of immune attack. Still further, the implantation process can cause some of these cells to rupture or lyse, which releases pro-inflammatory cytokines which induce T_{eff} cell migration to the site of inflammation.

Accordingly, there is a need to provide methods for implantation of transgenic beta cells into diabetic patients where these cells are immune protected for the entire period of implantation.

### REFERENCE TO DRAWINGS

FIGS. 1A and 1B illustrate LDH levels after incubation of genetically engineered human beta cells with PBMCs per Example 2.

FIG. 2 illustrates the expression levels of CXCL12 alpha and beta from genetically engineered human beta cells per Example 4.

FIGS. 3A and 3B illustrate LDH levels after incubation of senescent, genetically engineered human beta cells with PBMCs per Example 5.

### SUMMARY

As noted above, WO 2019/0100729 discloses genetically modified, allogeneic human beta cells that have been altered to express an effective amount of a fugetactic agent in the microenvironment surrounding these cells. However, removing these cells from an established medium where the microenviroment is protective means that immediately after implantation into a patient, these cells lack such a microenvironment and are susceptible to graft versus host immune response. In addition, the immediate aftermath of implantation induces an inflammatory environment which further induces immune cell intervention.

Described herein is a method for the in vivo implantation of allogeneic or xenogeneic mammalian cells genetically modified to express an effective amount of a fugetactic agent. This method entails the use of a rapidly degrading biocompatible polymer matrix or gel that is infused with an effective amount of the same fugetactic agent such that the matrix is masked to the immune system. The degradation period for the matrix is designed to allow the implanted cells time to regenerate their own protective microenvironment of the expressed fugetactic agent. As such, these methods are suitable for the direct implantation of these cells into the subject being treated.

Accordingly, in the methods provided herein, genetically engineered mammalian cells are implanted in vivo in combination into or with a biocompatible, biodegradable matrix that provides an exogenous source of a fugetactic agent in the microenvironment during the implantation period to inhibit immune cell destruction of said cells during implantation.

In one embodiment, there is provided a method for protecting genetically engineered, mammalian cells that express an effective concentration of a fugetactic agent that protects said cells against immune cell attack after implantation into a subject said method comprises:
a) selecting a cell or a population of mammalian cells that are genetically engineered to express an effective concentration of a fugetactic agent in the microenvironment;
b) implanting into a subject a rapidly biodegradable matrix or gel that is impregnated with and elutes an effective amount of the same fugetactic agent into the microenvironment surrounding the matrix or gel; and
c) implanting with or into said matrix or gel said cell or a population of said cells whereby said cells are protected from graft vs. host immune attack after implantation,
wherein said rapidly degrading biodegradable matrix or gel has a degradation period of about 10 days or less.

In one embodiment, the implanted cells are genetically engineered allogeneic or xenogeneic mammalian cells. In one embodiment, these cells are allogeneic human beta cells. In another embodiment, the cell or population of cells generate their own fugetactic microenvironment by no later than 10 days after implantation and preferably within 3 days.

In one embodiment, the matrix or gel comprises one or more components selected from oxygen releasing agents, glucose, an anti-inflammatory agent, and an antibiotic. The oxygen-releasing agents provide oxygen to the cells after implantation, whereas glucose is a cell nutrient. An anti-inflammatory agent can be employed to treat any inflammation arising from implantation, and an antibiotic can be employed to prevent infections at the site of implantation.

In one embodiment, the fugetactic agent is released into the site of implantation by elution from a biocompatible, biodegradable polymer that acts as the matrix or a gel. In one embodiment, the polymer is a cyanoacrylate having the formula CH₂═CH(C≡N)COOR, where R is an aliphatic group of 1 to 10 carbon atoms. The degradation time for such polymers is measured in hours or days based on the length of the aliphatic group. Longer aliphatic groups will have a longer degradation time. In one embodiment, a hydrogel containing sufficient water to avoid inducing osmotic shock at the site of implantation. In one embodiment, the hydrogel is selected from collagen/gelatin, chitosan, hyaluronic acid, chondroitin sulfate, alginate, agar/agarose, fibrin, polyethylene glycol, polyethylene oxide, polylactide, polyvinyl alcohol, and the like provided that the hydrogel has been a rapid degradation period in vivo.

Such rapidly biodegradable, biocompatible polymers are well known in the art. See, for example, Hahn, et al., International Journal of Biological Macromolecules, 40(4): 374-380 (2007). In addition, U.S. Pat. No. 8,846,022 discloses that an aqueous solution comprising 15% of PEG₃₃₅₀-Succinimidyl-Gluterate (“SG”) or [PEG₃₃₅₀-(SG)₂] derivatives cross-linked to polyethyleneimine (“PEI”) in a ratio of 15:1 PEG₃₃₅₀-(SG)₂ to PEI maintained at pH 7.4 and at 37° C. degrades in 4 days.

In one embodiment, a polymer is impregnated with a fugetactic agent in an amount sufficient that the microenvironment in and around the matrix is protective against immune cell penetration. In one embodiment, elution of the fugetactic agent from the biocompatible mass provides for immune protection over a period of time sufficient to allow implantation of the genetically modified cells. In one embodiment, the biocompatible and biodegradable polymer is selected to have an in vivo degradation period of from about 3 to about 10 days.

As used herein, the term “fugetactic agent” refers to any agent that, at sufficient concentrations, repels immune cells in vivo. Such fugetactic agents are known in the art. Examples of which are provided herein. For example, “CXCL12”, also known as “SDF-1”, is a cytokine (chemokine) produced by thymic and bone marrow stroma (see, e.g. U.S. Pat. No. 5,756,084, entitled: “Human stromal derived factor 1α. and 1β,” granted May 26, 1998, to Honjo, et al.). CXCL12 is known as C-X-C Motif Chemokine Ligand 12. CXCL12, at sufficient concentrations in the microenvironment surrounding cells, is known to repel effector T-cells while recruiting immune-suppressive regulatory T-cells to an anatomic site. See, e.g., Poznansky et al., Nature Medicine 2000, 6:543-8, which is incorporated herein by reference in its entirety. CXCL12 and its receptor CXCR4 are also reported to be an integral part of angiogenesis. There are numerous isoforms of CXCL12 that are suitable for use herein and are recited below. Preferred isoforms include CXCL12 alpha and CXCL12 beta.

Agents other than CXCL12 also are disclosed as being capable of repelling immune cells and therefore are fugetactic agents. These include, without limitation, gp120, other CXCR4 ligands, IL-8 (interleukin-8), CXCR4-binding antibodies, CXCL13, and the like. The choice of the specific fugetactic agent is not critical to the invention provided that the agent is used in sufficient amounts to impart immune protection to the implanted cells (e.g., engineered beta cells).

In one embodiment, there is provided a biologically compatible, implantable matrix comprising genetically engineered, immune protected, mammalian cells associated with said matrix and further wherein said cells express an effective amount of a fugetactic agents so as to resist destruction by human immune cells. In one embodiment, these cells are genetically engineered so that these cells express an effective amount of a fugetactic agent, preferably CXCL12, to provide immune protection after implantation. In one embodiment, the fugetactic agent is generated from a transgene for the agent (e.g., CXCL12) found in the cell.

In one embodiment, the mammalian cell is a human beta cell which is an allogenic cell or a non-human xenogeneic cell. Preferably, the cell is a beta cell that is allogenic. Such allogeneic or xenogeneic mammalian cells are subject to graft versus host immune responses resulting in immune cell attack and destruction if such cells are not masked to the immune system.

In one embodiment, the matrix comprising genetically engineered, immune protected, mammalian cells associated therewith further comprises an effective amount of a fugetactic agent that elutes from the matrix in sufficient amounts to provide initial immune protection to the cells after implantation into a subject. Such immune protection during the initial period immediately after cell implantation is required when the implanted cells require a period of time to express sufficient amounts of the fugetactic agent to provide for their own immune protection. In one embodiment, the eluting fugetactic agent is a CXCL12 isoform or a CXCL13 isoform. In one embodiment, the amount of fugetactic agent impregnated into the matrix is sufficient to maintain immune protection for the beta cells for up to about 10 days. Preferably, in such an embodiment, the implanted cells are allogenic beta cells.

In one embodiment, the implanted cells are genetically engineered human beta cells that are associated in a sufficient number with a single matrix or multiple matrices so as to control blood sugar of a patient lacking any control or inadequate control of blood sugar.

In one embodiment, the diabetes is diabetes type I (“T1D”). In another embodiment, the diabetes is type II (“T2D”).

The preferred matrices of this invention employ allogeneic human beta cells genetically engineered to express an effective amount of a fugetactic agent (e.g., CXCL12). Such engineered beta cells express a sufficient level of the fugetactic agent in the microenvironment surrounding the matrix to provide immune protection to the beta cells. Without wishing to be bound by theory, it is contemplated that this will allow sufficient time for the beta cells to survive and establish their own protective microenvironment and thereafter integrate with the blood and express insulin as necessary to maintain proper blood sugar levels in the subject.

### DETAILED DESCRIPTION

As noted above, this invention provides for matrices and methods for the implantation of mammalian cells into a patient. However, prior to describing this invention in detail, the following terms will first be defined.

### Definitions

The following terms have the following definitions. If a term is not defined, it has its accepted medical/biological definition.

The terminology used herein is for the purpose of describing particular embodiments only and is not intended to be limiting of the invention. As used herein, the singular forms “a,” “an,” and “the” are intended to include the plural forms as well, unless the context clearly indicates otherwise.

As used herein, the term “optional” or “optionally” means that the subsequently described event or circumstance can or cannot occur, and that the description includes instances where the event or circumstance occurs and instances where it does not.

As used herein, the term “about” when used before a numerical designation, e.g., temperature, time, amount, concentration, and such other, including a range, indicates approximations which may vary by (+) or (−) 15%, 10%, 5%, 1%, or any subrange or subvalue there between. Preferably, the term “about” when used with regard to a dose amount means that the dose may vary by +/−10%.

As used herein, the term “comprising” or “comprises” is intended to mean that the compositions and methods include the recited elements but not excluding others.

As used herein, the term “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention.

As used herein, the term “consisting of” shall mean excluding more than trace elements of other ingredients and substantial method steps. Embodiments defined by each of these transition terms are within the scope of this invention.

The term “implantation” or “implantation process” includes the period from initial site preparation for depositing genetically modified cells as described herein up to the point where the implanted cells are immune protected by virtue of their expression of an effective amount of a fugetactic agent. This period can extend up to several days and perhaps as long as 10 days. It is understood that, in some cases, engineered allogeneic cells immediately deposited into/onto the implantation site in a subject may not initially create a microenvironment that is effective in providing immune protection to these cells. In such cases, these cells will, over a period of no more than 10 days, express sufficient fugetactic agent so as to become immune protected. The lag period between deposition and these cells becoming immune protected by their expression of sufficient fugetactic agent is included within the implantation period.

The term “into the matrix or gel” means that the genetically engineered mammalian cells are deposited into a matrix or gel that has already been implanted into the subject. It is understood that cells are typically are implanted immediately after implantation of the gel or matrix or within a short period of time thereafter (e.g., less than 2 hours) to allow the gel or matrix to equilibrate in the physiological environment. In this embodiment, separate implantation of the cells is done in order to limit the stress on the cells and to achieve better survival rates. In one embodiment, the gel or matrix contains a biocompatible label such as a fluorescent label (e.g., fluorescein) that allows the clinician to target the implantation of the cells directly into the gel or matrix. Alternatively, the to-be implanted cells can be incorporated into the matrix or gel prior to implantation and then implanted into the subject together.

In one embodiment, the source of a fugetactic agent is a population of genetically engineered cells that express a fugetactic agent such as CXCL12. One set of genetically engineered cells useful in such methods includes adipose cells that are genetically modified to express a fugetactic agent.

In one embodiment, the genetically modified adipose cells are placed into a biologically compatible matrix that is preferably but not necessarily non-biodegradable and can be placed in the microenvironment adjacent to non-engineered beta cells. The genetically modified adipose cells act as companion cells for human beta cells differentiated from a human stem cell. Such microenvironments include those disclosed in diabetesresearchconnection.org/exploring-beta-cell-regeneration-treating-type-1-diabetes/which is incorporated herein by reference in its entirety.

In one embodiment, the source of a fugetactic agent is a biologically compatible matrix or gel that has been impregnated with a fugetactic agent which elutes from said matrix or gel at a fugetactic concentration.

The term “biologically compatible, biodegradable implantable matrix or gel” refers to any matrix or gel that is biologically compatible with the subject to which it is to be implanted such that the subject does not experience any substantial adverse biological response that would render its use unacceptable. Such matrices are rapidly biodegradable under physiological conditions to maintain an immune repellent environment (fugetactic barrier) surrounding the implanted cells. Suitable matrices include those cited herein. As used herein, the term “rapidly biodegradable” means that at least 50% of the matrix or gel biodegrades within 10 days, or at least 70% of the matrix or gel biodegrades within 10 days; or at least 90% of the matrix or gel biodegrades with 10 days. In all cases, the percent is a volume percent that can be measured in vitro under simulated physiological conditions well known in the art.

The term “genetically engineered, immune protected cells” refers to mammalian cells (e.g., human beta cells) that express sufficient amounts of a fugetactic agent so as to resist immune cell attack as well as be capable of controlling blood sugar in the subject to which they are to be implanted.

The term “fugetactic barrier” refers to the microenvironment surrounding an implanted cell that expresses or contains a sufficient amount of a fugetactic agent such that the concentration of said agent in said microenvironment is capable of repelling immune cells.

The term “associated therewith,” as it relates to mammalian cells and an implantable matrix, refers to genetically modified cells as described herein which are embedded in the matrix, found on the surface of the matrix or substantially surrounded by the matrix in any manner such that the cells are resistant to cell death in the presence of human immune cells.

The term “lacking control of blood sugar” refers to diabetic patients that do not express insulin or proinsulin in amounts that can control blood sugar. Such diabetic patients are typically type 1 diabetic patients.

The term “lacking inadequate control of blood sugar” refers to diabetic patients that do express insulin or proinsulin but not in amounts that control blood sugar to levels that are considered to be physiologically appropriate (e.g., AB1Ac levels of less than 7). Such diabetic patients are typically type 2 diabetic patients.

The term “detectable component” refers to any component attached to or integrated into the matrix so as to permit detection of the matrix. Suitable detectable components include fluorescent compounds well known in the art such as fluorescein, metal components that are visible under fluoroscopy such as tantalum, gold, etc. The choice of the detectable component is not critical and is selected relative to its ease of incorporation and its compatibility to the material comprising the matrix.

### Methods

In one embodiment, this invention provides for methods that employ genetically engineered mammalian cells that, after the implantation into a subject are capable of immune protection and control of the patient's blood sugar levels. Such immune protected cells are those that express a sufficient amount of a fugetactic agent after implantation so as to be immune protected.

The methods described herein further comprise implantation of such cells into or with a matrix or gel that elutes a fugetactic agent to impart immune protection up to 10 days after implantation. Without being limited to any theory, immediately upon deposition of the genetically modified cells into a subject, the concentration of the fugetactic agent in the microenvironment of the implanted cells may be insufficient to repel immune cells. If the cells are allogeneic or xenogeneic, such will result in a graft versus (vs.) host immune response that will kill some or all of these cells. This invention addresses this possibility by accompanying implantation with direct or indirect administration of an effective amount of a fugetactic agent. This creates a microenvironment around these cells that immune protects them during implantation. Once the cells are able to generate a fugetactic microenvironment of their own, the need for exogenous (not produced by the implanted cells) fugetactic agent is no longer required. Hence, the use of a biodegradable polymer in the matrix is preferred as it will be removed from the body.

In one embodiment, direct administration of a fugetactic agent to the site of implantation is merely providing a solution, suspension, or injectable fluid comprising a high concentration of the fugetactic agent to that site. Such a solution, suspension or injectable solution can have a high viscosity so as to minimize movement of the injected material from the site of implantation. However, in a preferred embodiment, indirect administration is conducted using fugetactic eluting biodegradable matrix that elute fugetactic agents, and particles impregnated with the fugetactic agent. In such cases, sufficient amounts of the fugetactic agent are provided to the implantation site such that the deposited cells are immune protected during the implantation process.

In one embodiment, the implanted mammalian cells are functional beta cells that are genetically modified to express an effective amount of a fugetactic agent after implantation. In one preferred embodiment, such cells have been induced into senescence (the inability to replicate) by exposure to one of many different methods well known in the art. Suitable methods include contacting these cells with one or more of the following mitomycin C, agents that induce telomere dysfunction due to replication-associated telomere shortening, and subcytoxic stresses such as exposure to UV (ultraviolet light), gamma irradiation, hydrogen peroxide, and hyperoxia. The specific means by which these beta cells are rendered non-replicative is not critical provided that these cells can be implanted and maintain functionality without the risk of further differentiation coupled with cellular division.

One aspect of the invention is a method of treating diabetes in a subject in need thereof, comprising administering to the subject autologous, genetically modified beta cells that comprise a transgene encoding a fugetactic agent (e.g., a CXCL12 isoform) or have been genetically modified to express an endogenous fugetactic agent (e.g., a CXCL12 isoform) in fugetactic amounts.

An aspect of this invention are genetically modified beta cells, e.g., human autologous beta cells or non-autologous beta cells, e.g., allogeneic beta cells, comprising a nucleic acid encoding a fugetactic agent (e.g., CXCL12) in operable linkage with a promoter, such that the fugetactic agent (e.g., CXCL12) is expressed at a fugetactic level in the beta cell microenvironment. The promoter may be a promoter endogenous to the beta cell or heterologous to but functional in the beta cell. Preferably, the nucleic acid encoding the fugetactic agent (e.g., CXCL12) is endogenous to the subject being treated with the transgenic beta cells.

An aspect of this invention are beta cells comprising a genetically modified endogenous gene encoding a fugetactic agent (e.g., CXCL12) wherein the gene is modified to comprise a heterologous promoter in operable linkage with the fugetactic agent-encoding sequence, such that the fugetactic agent is expressed from the endogenous gene at a fugetactic level in the beta cell microenvironment. The promoter may be introduced into the beta cells to be in operable linkage with the fugetactic agent-encoding sequence using genome editing techniques known in the art.

In general, this invention provides for beta cells that express a fugetactic agent (e.g., CXCL12) at a level sufficient to block or inhibit migration of immune cells to the beta cells or sufficient to repel immune cells. The terms immune cells and mononuclear cells (T-cells, B-cells, and NK cells) may be used interchangeably. The ability of a fugetactic agent (e.g., CXCL12) polypeptide to repel immune cells (e.g., effector T-cells) can be assessed in vitro, using a boyden chamber assay. See, e.g., as previously described in Poznansky et al., Journal of Clinical Investigation, 109, 1101 (2002).

Without wishing to be bound by any theory, Applicant contemplates that in an aspect of this invention the amount of fugetactic agent (e.g., CXCL12) produced by a genetically modified cells are sufficient to provide a fugetactic effect in the microenvironment where the cells are implanted. However, the amount of fugetactic agent generated is insufficient to raise the systemic levels of the agent and upset the balance between the beneficial effects of the agent in one process while producing deleterious consequences in another. In addition, CXCL12 is known to induce angiogenesis when bound to its receptor CXCR4. Again, without being bound by any theory, it is contemplated that the microenvironment of the implanted transgenic beta cells expressing CXCL12 will induce an angiogenic response that enhance the survivability of the implanted cells.

In one embodiment, the matrices of this invention can employ oxygen releasing components that provide for molecular oxygen to the cells so as to enhance cell survival rates. Oxygen releasing component are known in the art and include those set forth in www.cell.com/trends/biotechnology/fulltext/S0167-7799(16)30058-0, which is incorporated herein by reference in its entirety. Also, see Ozcelik, et al., https://doi.org/10.1016/j.ijpharm.2021.120810. In another embodiment, the matrices or gels can incorporate glucose as a nutrient for the cells. See Hadler, J. Biol. Chem., 255(8): 3532-3535 (1980).

The fugetactic effective amount of a fugetactic agent (e.g., CXCL12) is any amount sufficient to block immune cell homing to the beta cells or in some aspects repel the immune cells from the beta cells. For example, a fugetactic effective amount of fugetactic agent (e.g., CXCL12) in the transgenic beta cell microenvironment from about 100 ng/ml to about 200 ng/ml, from about 100 ng/ml to about 300 ng/ml, from about 100 ng/ml to about 400 ng/ml, from about 100 ng/ml to about 500 ng/ml, from about 100 ng/ml to about 600 ng/ml, from about 100 ng/ml to about 700 ng/ml, from about 100 ng/ml to about 800 ng/ml, from about 100 ng/ml to about 900 ng/ml, or from about 100 ng/ml to about 1 ug/ml. Preferably the amount of the fugetactic agent (e.g., CXCL12) in the beta cell microenvironment is a fugetactic sufficient amount that preferably ranges from about 20 ng/ml to 1 ug/ml or more preferably from about 100 ng/ml to about 500 ng/ml. Without wishing to be bound by theory, it is contemplated that the transgenic beta cells may express sufficient amounts of the fugetactic agent such that the microenvironment creating the fugetactic effect extends to adjacent to non-transgenic beta cells.

Although mice and mouse DNA are widely used by immunologists to gain insight into the workings of the human immune system, there are significant differences between humans and mice. Accordingly, the fugetactic agent (e.g., CXCL12) encoded by the vector is preferably a human agent. Javier Mestas & Christopher C. W. Hughes, “Of Mice and Not Men: Differences between Mouse and Human Immunology,” Journal of Immunology 2004, 172(5), 2731-2738; O. Cabrera et al., “The unique cytoarchitecture of human pancreatic islets has implications for islet cell function,” PNAS 2006, 103(7), 2334-2339; M. Votey, “Of mice and men: how the nPOD program is changing the way researchers study type 1 diabetes,” diaTribe, Network for Pancreatic Organ Donors with Diabetes (Aug. 21, 2015).

CXCL12 polypeptides are known in the art. See, e.g., Poznansky et al., Nature Medicine 2000, 6:543-8 and US Publ. 20170246250 incorporated herein by reference in their entirety. The terms CXCL12 and SDF-1 may be used interchangeably. Exemplary CXCL12/SDF1 Isoforms are provided in Table I of U.S. Publ. 20170246250. Exemplary CXCL12/SDF1 Isoforms are also provided in Table 1 (below):

TABLE 1
HUMAN CXCL12/SDF1 ISOFORMS

| | | Accession | |
|---|---|---|---|
| | Accession | Number | |
| Name | Number | Versions | Sequence |
| SDF-1 | NP_954637 | NP_954637.1 | MNAKVVVVLV LVLTALCLSD |
| Alpha | | GI: 40316924 | GKPVSLSYRC PCRFFESHVA |
| | | | RANVKHLKIL NTPNCALQIV |
| | | | ARLKNNNRQV CIDPKLKWIQ |
| | | | EYLEKALNK (SEQ ID NO: 1) |
| SDF-1 | P48061 | P48061.1 | MNAKVVVVLV LVLTALCLSD |
| Beta | | GI: 1352728 | GKPVSLSYRC PCRFFESHVA |
| | | | RANVKHLKIL NTPNCALQIV |
| | | | ARLKNNNRQV CIDPKLKWIQ |
| | | | EYLEKALNKR FKM (SEQ ID NO: 2) |
| SDF-1 | NP_001029058 | NP_001029058.1 | MNAKVVVVLV LVLTALCLSD |
| Gamma | | GI: 76563933 | GKPVSLSYRC PCRFFESHVA |
| | | | RANVKHLKIL NTPNCALQIV |
| | | | ARLKNNNRQV CIDPKLKWIQ |
| | | | EYLEKALNKG RREEKVGKKE |
| | | | KIGKKKRQKK RKAAQKRKN (SEQ |
| | | | ID NO: 3) |
| SDF-1 | | Yu et al. | MNAKVVVVLV LVLTALCLSD |
| Delta | | Identification | GKPVSLSYRC PCRFFESHVA |
| | | and expression | RANVKHLKIL NTPNCALQIV |
| | | of novel | ARLKNNNRQV CIDPKLKWIQ |
| | | isoforms of | EYLEKALNNL ISAAPAGKRV |
| | | human stromal | IAGARALHPS PPRACPTARA |
| | | cell-derived | LCEIRLWPPP EWSWPSPGDV (SEQ |
| | | factor 1. Gene | ID NO: 4) |
| | | (2006) vol. 374 | |
| | | pp. 174-9 | |
| SDF-1 | | Yu et al. | MNAKVVVVLV LVLTALCLSD |
| Epsilon | | Identification | GKPVSLSYRC PCRFFESHVA |
| | | and expression | RANVKHLKIL NTPNCALQIV |
| | | of novel | ARLKNNNRQV CIDPKLKWIQ |
| | | isoforms of | EYLEKALNNC (SEQ ID NO: 5) |
| | | human stromal | |
| | | cell-derived | |
| | | factor 1. Gene | |
| | | (2006) vol. 374 | |
| | | pp. 174-9 | |
| SDF-1 | | Yu et al. | MNAKVVVVLV LVLTALCLSD |
| Phi | | Identification | GKPVSLSYRC PCRFFESHVA |
| | | and expression | RANVKHLKIL NTPNCALQIV |
| | | of novel | ARLKNNNRQV CIDPKLKWIQ |
| | | isoforms of | EYLEKALNKI WLYGNAETSR (SEQ |
| | | human stromal | ID NO: 6) |
| | | cell-derived | |
| | | factor 1. Gene | |
| | | (2006) vol. 374 | |
| | | pp. 174-9 | |

In one embodiment, a CXCL12 polypeptide has at least about 85%, 90%, 92%, 95%, 96%, 97%, 98%, 99%, or 100% amino acid sequence identity to NP 001029058 and has chemokine or fugetaxis activity. In one embodiment, a CXCL12 polypeptide has at least about 85%, 90%, 92%, 95%, 96%, 97%, 98%, 99%, or 100% amino acid sequence identity to SEQ ID NO: 1, SEQ ID NO: 2, SEQ ID NO: 3, SEQ ID NO: 4, SEQ ID NO: 5, or SEQ ID NO: 6, and has chemokine or fugetaxis activity. Such sequence identity is based on the replacement of a first amino acid with a known conservative second amino acid. Such conservative replacements are well established in the art and the testing of the resulting modified CXCL12 polypeptide for its fugetactic properties are well known in the art. See, for example, Poznansky, supra.

The transgenic beta cells used in the methods described herein may be autologous or non-autologous, e.g., allogenic beta cells. “Autologous” cells are cells from the same individual. “Allogeneic” cells are cells from a genetically similar but not identical a donor of the same species. Allogenic cells useful in the methods of this invention maybe from a human subject. Allogenic cells useful in the methods of this invention maybe from a relative, e.g., a sibling, a cousin, a parent, or a child, or a non-relative. Criteria for selecting an allogenic donor are well known in the art, e.g., Tatum et al., Diabetes Metab. Syndr. Obes. (2017) 10: 73-78. Human allogeneic beta cells are commercially available, and autologous beta cells are produced by the methods described by Egli, et al., supra.

In an embodiment, the transgenic beta cells used in the methods of this invention are autologous transgenic beta cells that can be prepared by deriving beta cells from multipotent progenitor cells or pluripotent stem cells obtained from the patient by methods known in the art. These derived beta cells may be transfected with a vector comprising a nucleic acid sequence encoding the fugetactic agent (e.g., CXCL12).

Alternatively, the transgenic beta cells used in the methods of this invention may be prepared by isolating islet beta cells from the subject in need thereof. These isolated islet beta cells may be transfected with a vector comprising a nucleic acid sequence encoding the fugetactic agent (e.g., CXCL12). Alternatively, the beta cell may be genetically modified to express the endogenous fugetactic agent (e.g., CXCL12) gene such that it constitutively produces a fugetactic effective amount of the fugetactic agent (e.g., CXCL12).

In an embodiment, the beta cells are transfected with an expression vector comprising a nucleic acid molecule that encodes the fugetactic agent (e.g., CXCL12), said nucleic acid molecule being in operable linkage with a promoter suitable for expression in the beta cells. The vector may integrate into the genome of the beta cell, or it may exist episomally and not integrate into the genome.

The transgenic beta cells of the invention may also be prepared from an adult stem cell by isolating adult stem cells from the subject, culturing the stem cells under appropriate conditions to expand the population and to induce differentiation into beta cells. The cells may be modified to express fugetactic effective amounts of the fugetactic agent (e.g., CXCL12) by transfecting the cells with an expression vector encoding fugetactic amounts of the fugetactic agent (e.g., CXCL12) or by editing the genome to express fugetactic amounts of the fugetactic agent (e.g., CXCL12). The vector may be introduced into the stem cells prior to differentiation into beta cells, or the genome of the stem cells may be edited to contain the heterologous promoter. Alternatively, the vector may be introduced into the resulting beta cells, or the genome of the resulting beta cells may be edited to contain the heterologous promoter.

The transgenic beta cells of the invention may also be prepared by generating induced pluripotent stem (iPS) cells from somatic cells, e.g., beta cells, of a subject; treating the iPS cells to induce differentiation into beta cells; and transfecting the differentiated beta cells with a vector comprising a nucleic acid sequence encoding the fugetactic agent (e.g., CXCL12).

The transgenic beta cells of the invention may also be prepared by preparing induced pluripotent stem (iPS) cells generated from somatic cells of a subject; transfecting the iPS cells with a vector comprising a nucleic acid sequence encoding the fugetactic agent (e.g., CXCL12); and treating the iPS cells, before or after transfection, to induce differentiation into beta cells.

The transgenic beta cells of this invention may also be generated by obtaining progenitor cells or progenitor-like cells, e.g., pancreatic β-cell progenitors, introducing a vector comprising a nucleic acid sequence encoding the fugetactic agent (e.g., CXCL12) into the cells, and treating the cells either before or after introducing the vector to induce differentiation into beta cells, or insulin releasing cells responsive to glucose levels in the body, by methods known in the art, see e.g., Millman et al., Nature Communications (10 May 2016) page 1-8); Baek et al. Curr. Stem Cell Rep. (2016) 2:52-61; Russ et al., EMBO. J. 34, 1759-1772 (2015); and, Qadir et al., Cell Reports 22, 2408-2420 (Feb. 27, 2018). The progenitor cell and progenitor-like cells may be autologous or non-autologous, e.g., allogeneic, to the subject treated with the transgenic cells. Insulin-producing cells responsive to glucose levels in the body (see, e.g., Qadir et al., supra), may be genetically modified as described herein to express fugetactic levels of the fugetactic agent (e.g., CXCL12) and are also an embodiment of this invention. Such genetically modified insulin producing cells can likewise be used in the methods of this invention to treat diabetes as described herein.

Any suitable somatic cell from a subject may be reprogrammed into an iPS cell by methods known in the art, see, e.g., Pagliuca and Melton (2013) How to make a functional β-cell, Development (2013) 140(12): 2472-2483; Yu et al. (2007). Induced pluripotent stem cell lines derived from human somatic cells. Science 318, 1917-1920; Takahashi and Yamanaka, 2006, Cell 126(4): 663-676; Wernig et al., 2007, Nature 448:7151; Okita et al., 2007 Nature 448: 7151; Maherali et al., 2007 Cell Stem Cell 1:55-70; Lowry et al., 2008 PNAS 105:2883-2888; Park et al., 2008 Nature 451: 141-146; Takahashi et al., 2007 Cell 131, 861-872; U.S. Pat. Nos. 8,546,140; 7,033,831; and 8,268,620. The iPS cells may be differentiated into beta cells using methods known in the art, see, e.g., U.S. patent publication no. 20170081641 and U.S. patent publication no 20130164787, and Millette and Georgia, “Gene Editing and Human Pluripotent Stem Cells: Tools for advancing Diabetes Disease Modeling and Beta Cell Development”, Current Diabetes Reports November 2017, 17: 116; U.S. patent application no. 20130273651; Shi, Y., et al., Stem Cells, 25: 656-662 (2005); or Tateishi, K., et al., J Biol Chem., 283: 31601-31607 (2008).

Preferably the fugetactic agent-encoding sequence is in operable linkage with a regulatory region that is suitable for expression in a beta cell. Suitable regulatory regions are known in the art and include promoters such as, e.g., mammalian promoters including, e.g., hypoxanthine phosphoribosyl transferase (HPTR), adenosine deaminase, pyruvate kinase, β-actin promoter, muscle creatine kinase promoter, and human elongation factor promoter (EF1α), a GAPDH promoter, an actin promoter, and a ubiquitin promoter and viral promoters including SV40 early promoter, SV40 late promoter, metallothionein promoter, murine mammary tumor virus promoter, Rous sarcoma virus promoter, polyhedrin promoter, human immunodeficiency virus (HIV) promoters, cytomegalovirus (CMV) promoters, adenoviral promoters, adeno-associated viral promoters, or the thymidine kinase promoter of herpes simplex virus. Other bacterial, viral, and eukaryotic promoters are also well known in the art (see, e.g., in Sambrook and Russell (Molecular Cloning: a laboratory manual, Cold Spring Harbor Laboratory Press). The regulatory region in operable linkage with the fugetactic agent-encoding sequence may be any constitutive promoter suitable for expression in the subject's cells.

The transgenic cells expressing the fugetactic agent (e.g., CXCL12) of this invention, whether autologous or non-autologous, e.g., allogeneic, may be administered to a subject in need thereof by any means known in the art for administering beta cells. The transgenic cells of this invention may be administered in an amount sufficient to provide levels of insulin able to alleviate at least some of the symptoms associated with low levels of insulin.

Another aspect of the invention is a method of treating diabetes in a subject in need thereof, comprising the steps of: (a) obtaining or deriving beta cells or insulin-producing beta-like cells, from the subject; (b) introducing a suitable expression vector encoding the fugetactic agent (e.g., CXCL12) into the cells to form autologous transgenic cells expressing the introduced the fugetactic agent (e.g., CXCL12); and (c) transplanting the autologous transgenic cells into the subject.

Many vectors useful for transferring exogenous genes into mammalian cells, e.g., beta cells, including vectors that integrate into the genome and vectors do not integrate into the genome but exist as episomes, and methods for introducing such vectors into cells are available and known in the art. For example, retroviral vectors, lentiviral vectors, adenoviral vectors, adeno-associated (AAV)-based vectors, and EBV-based vectors may be used. See, e.g., US 20110280842, Narayanavari and Izsvák, Cell Gene Therapy Insights 2017; 3(2), 131-158; Hardee et al., Genes 2017, 8, 65; Tipanee et al., Bioscience Reports (2017) 37, and Chira et al. Oncotarget, Vol. 6, No. 31, pages 30675-30703.

Another aspect of the invention is a method for promoting survival of beta cells in a biological sample comprising immune cells comprising introducing an expression vector encoding the fugetactic agent (e.g., CXCL12) into the beta cells, or by editing the genome of the beta cells such that the beta cells express fugetactic amounts of the fugetactic agent (e.g., CXCL12). In an aspect of this invention, the fugetactic agent (e.g., CXCL12) is expressed by the beta cells at a level sufficient to block or inhibit migration of immune cells, e.g., T-cells, B-cells, and/or NK cells, to the beta cells. In an aspect of this invention, the fugetactic agent (e.g., CXCL12) is expressed by the beta cells at a level sufficient to repel the immune cells from the beta cells. In an aspect of this invention, the genetically modified beta cells are in a subject, e.g., a human subject having Type 1 or Type 2 diabetes. The beta cells are preferably autologous beta cells of the subject.

Methods for the delivery of viral vectors and non-viral vectors to mammalian cells are well known in the art and include, e.g., lipofection, microinjection, ballistics, virosomes, liposomes, immunoliposomes, polycation or lipid-nucleic acid conjugates, naked DNA, artificial virions, and agent-enhanced uptake of DNA. Lipofection reagents are sold commercially (e.g., Transfectam™ and Lipofectin™). Cationic and neutral lipids suitable for efficient receptor-recognition lipofection of polynucleotides are known. Nucleic acid can be delivered to cells (ex vivo administration) or to target tissues (in vivo administration). The preparation of lipid:nucleic acid complexes, including targeted liposomes such as immunolipid complexes, is well known to those of skill in the art. Recombination-mediated systems can be used to introduce the vectors into the cells. Such recombination methods include, e.g., the use of site-specific recombinases like Cre, Flp or PHIC31 (see e.g. Oumard et al., Cytotechnology (2006) 50: 93-108) which can mediate directed insertion of transgenes.

Vectors suitable for use in this invention include expression vectors comprising a nucleic acid encoding a fugetactic agent (e.g., CXCL12) in operable linkage with a promoter to direct transcription. Suitable bacterial and eukaryotic promoters are well known in the art and described, e.g., in Sambrook and Russell (Molecular Cloning: a laboratory manual, Cold Spring Harbor Laboratory Press). The promoter used to direct expression of the fugetactic agent (e.g., CXCL12) may be, e.g., example, SV40 early promoter, SV40 late promoter, metallothionein promoter, murine mammary tumor virus promoter, Rous sarcoma virus promoter, polyhedrin promoter, or other promoters shown to be effective for expression in mammalian cells.

Vectors useful in the methods of this invention include, e.g., SV40 vectors, papilloma virus vectors, Epstein-Barr virus vectors, retroviral vectors, and lentiviral vectors (e.g., self-inactivating lentiviral vectors).

The vectors used in this invention may comprise regulatory elements from eukaryotic viruses, e.g., SV40, papilloma virus, and Epstein-Barr virus, including, e.g., signals for efficient polyadenylation of the transcript, transcriptional termination, ribosome binding, and/or translation termination. Additional elements of the vectors may include, e.g., enhancers, and heterologous spliced intronic signals.

In an embodiment of this invention, the genome of the beta cell may be genetically modified to increase the expression levels of an endogenous fugetactic agent (e.g., CXCL12) gene. Such increased expression may be achieved by introducing a heterologous promoter in operable linkage with the endogenous fugetactic agent (e.g., CXCL12) gene or by altering the endogenous fugetactic agent (e.g., CXCL12) promoter such that the beta cell expresses a fugetactic level of fugetactic agent (e.g., CXCL12). Such increased expression may be achieved by introducing a promoter into the genome of the beta cell. The promoter is in operable linkage with the endogenous fugetactic agent-encoding sequence and thereby expresses or overexpresses the fugetactic agent in a fugetactic amount.

Gene editing technologies for modifying the genome are well known in the art and include, e.g. CRISPR/CAS 9, Piggybac, Sleeping Beauty genome editing systems, (see for example., Zhang et al. Molecular Therapy Nucleic Acids, Vol 9, December 2017, page 230-241; systems (see, e.g., Cong et al., Science. 2013; 339(6121): 819-23; Mali et al., Science. 2013; 339(6121): 823-6; Gonzalez et al., Cell Stem Cell. 2014; 15(2): 215-26); He et al., Nucleic Acids Res. 2016; 44(9); Hsu et al., Cell. 2014; 157(6): 1262-78.), zinc finger nuclease-based systems (see, e.g., Porteus and Carroll, Nat Biotechnol. 2005; 23(8): 967-73; Urnov et al., Nat. Rev Genet. 2010; 11(9): 636-46), TALEN-based systems (transcription activator-like effector nucleases)(see e.g., Cermak et al., Nucleic Acids Res. 2011; 39(12); Hockemeyer et al., Nat Biotechnol. 2011; 29(8): 731-4; Joung and Sander J D, Nat Rev Mol Cell Biol. 2013; 14(1): 49-55; Miller et al., Nat Biotechnol. 2011; 29(2): 143-8, and Reyon et al., Nat Biotechnol. 2012; 30(5): 460-5).

Another aspect of the invention is a method of modulating the levels of insulin in a subject, comprising administering to the subject in need thereof the beta cells of this invention wherein the beta cells express insulin and produce a fugetactic agent (e.g., CXCL12) in a fugetactic amount. The beta cells may be autologous beta cells or non-autologous beta cells, e.g., allogeneic beta cells, and may harbor a vector expressing the fugetactic agent, which vector may be integrated into the beta cell genome or exist episomally. In an embodiment of this invention the transgenic beta cells may be a genetically modified to overexpress endogenous fugetactic agent (e.g., CXCL12) at a fugetactic level.

Methods of introducing (implanting) the transgenic beta cells described herein into individuals are well known to those of skills in the art and include, but are not limited to, injection, intravenous, intraportal, or parenteral administration. Single, multiple, continuous or intermittent administration can be effected. See e.g., Schuetz and Markmann, Curr Transplant Rep. 2016 September; 3(3): 254-263.

Pharmaceutically acceptable carriers include sterile aqueous solutions or dispersions and sterile powders for the extemporaneous preparation of sterile injectable solutions or dispersion. The use of media and agents for pharmaceutically active substances, including cells, is well known in the art. A typical pharmaceutical composition for intravenous infusion of beta cells could be made up to contain 250 ml of sterile Ringer's solution, and 100 mg of the combination. Actual methods for preparing parenterally administrable compounds will be known or apparent to those skilled in the art and are described in more detail in, for example, REMINGTON'S PHARMACEUTICAL SCIENCE, 17th ed., Mack Publishing Company, Easton, Pa. (1985), and the 18th and 19th editions thereof, which are incorporated herein by reference.

The transgenic and genetically modified beta cells of the invention can be introduced into any of several different sites well known in the art, including but not limited to the pancreas, the abdominal cavity, the kidney, the liver, the celiac artery, the portal vein or the spleen of the subject.

The transgenic and genetically modified beta cells may be transplanted into the subject via a graft. An ideal beta cell transplantation site would be one that supports the long-term function and survival of grafted cells in the subject and is easily accessible for maximal patient safety.

The term “effector T-cell” refers to a differentiated T-cell capable of mounting a specific immune response by releasing cytokines.

The term “regulatory T-cell” refers to a T-cell that reduces or suppresses the immune response of B-cells or other T-cells to an antigen.

The terms “type 1 diabetes” and “type 2 diabetes” refer to two major pathophysiologies related to increasing glycemia. The first is an autoimmune attack against the pancreatic insulin-producing beta-cells (Type 1 diabetes) whilst the second is associated to poor beta-cell function and increased peripheral insulin resistance (Type 2 diabetes). Similar to Type 1, beta-cell death is also observed in Type 2 diabetes. Type 1 and often Type 2 diabetes requires the person to inject insulin. Type 1 diabetes is typically characterized by loss of the insulin-producing beta-cells of the islets of Langerhans in the pancreas leading to insulin deficiency. This type of diabetes can be further classified as immune-mediated or idiopathic. The majority of Type 1 diabetes is of the immune-mediated nature, where beta-cell loss is a T-cell mediated autoimmune attack. Type 2 diabetes is characterized by beta-cell dysfunction in combination with insulin resistance. The defective responsiveness of body tissues to insulin is believed to involve the insulin receptor. Similar to Type 1 diabetes an insufficient beta cell mass is also a pathogenic factor in many Type 2 diabetic patients. In the early stage of Type 2 diabetes, hyperglycemia can be reversed by a variety of measures and medications that improve insulin secretion and reduce glucose production by the liver. As the disease progresses, the impairment of insulin secretion occurs, and therapeutic replacement of insulin may sometimes become necessary in certain patients.

A “subject” or “patient” refers to a mammal, preferably to a human subject.

A “subject in need thereof” or “patient in need thereof” is a subject having Type I or Type II diabetes.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition, or symptoms associated therewith be completely eliminated.

All of the references cited herein are incorporated by reference in their entirety.

### EXAMPLES

### Example 1: Preparation of Transgenic Beta Cells

Pancreatic beta cells derived from human induced pluripotent stem cells were purchased from Takara Bio U.S.A., Inc. (Mountain View, CA) and cultured according to provided instructions.

Cells were transduced with lentiviral vectors (pLenti-C-Myc-DDK, OriGene Technologies, Rockville, MD) containing a human CXCL12 isotype (CXCL12a/SDF-1alpha or CXCL12b/SDF-1beta) or control. The sequences, including the tag (underlined) are provided below. The concentration of the CXCL12 isotype was determined by ELISA (RayBioTech, Norcross, GA).

CXCL12a (aka SDF1a)
Accession No. NM_199168
SEQ ID NO.: 7
ATGAACGCCAAGGTCGTGGTCGTGCTGGTCCTCGTGCTGA
CCGCGCTCTGCCTCAGCGACGGGAAGCCCGTCAGCCTGAG
CTACAGATGCCCATGCCGATTCTTCGAAAGCCATGTTGCC
AGAGCCAACGTCAAGCATCTCAAAATTCTCAACACTCCAA
ACTGTGCCCTTCAGATTGTAGCCCGGCTGAAGAACAACAA
CAGACAAGTGTGCATTGACCCGAAGCTAAAGTGGATTCAG
GAGTACCTGGAGAAAGCTTTAAACAAGACGCGTACGCGGC
CGCTCGAGCAGAAACTCATCTCAGAAGAGGATCTGGCAGC
AAATGATATCCTGGATTACAAGGATGACGACGATAAGGTT
TAA
CXCL12b (aka SDF1b)
Accession No. NM_000609
SEQ ID NO.: 8
ATGAACGCCAAGGTCGTGGTCGTGCTGGTCCTCGTGCTGA
CCGCGCTCTGCCTCAGCGACGGGAAGCCCGTCAGCCTGAG
CTACAGATGCCCATGCCGATTCTTCGAAAGCCATGTTGCC
AGAGCCAACGTCAAGCATCTCAAAATTCTCAACACTCCAA
ACTGTGCCCTTCAGATTGTAGCCCGGCTGAAGAACAACAA
CAGACAAGTGTGCATTGACCCGAAGCTAAAGTGGATTCAG
GAGTACCTGGAGAAAGCTTTAAACAAGAGGTTCAAGATGA
CGCGTACGCGGCCGCTCGAGCAGAAACTCATCTCAGAAGA
GGATCTGGCAGCAAATGATATCCTGGATTACAAGGATGAC
GACGATAAGGTTTAA

### Example 2: Transgenic Beta Cells Repel PBMCs

The transgenic beta cells from Example 1 were contacted with human peripheral blood mononuclear cells (PBMCs, Innovative Research, Novi, MI) at a ratio of 30:1 (PBMCs to beta cell). Briefly, PBMCs were resuspended in beta full culture medium, counted, and adjusted to allow for a 30:1 PBMC:beta cell ratio with the addition of 100 uL of PBMCs (to minimize dilution of the expressed CXCL12). The final volume was 1.1 mL. Background controls of beta cells without PBMCs and PBMCs without beta cells were also created. Immediately 150 uL of medium was removed from each sample and centrifuged at 1200×g for 10 minutes. Supernatant was removed and stored at 4° C. (time zero). Cells were returned to the incubator and sampled in a similar way to the time zero sample at both 24 and 48 hours later.

Release of lactate dehydrogenase (LDH) was tested at 24 and 48 hours after contact using Pierce LDH Cytotoxicity Assay Kit (Thermo Scientific) according to manufacturer's instructions. LDH is an indicator of cytotoxicity (cell lysis).

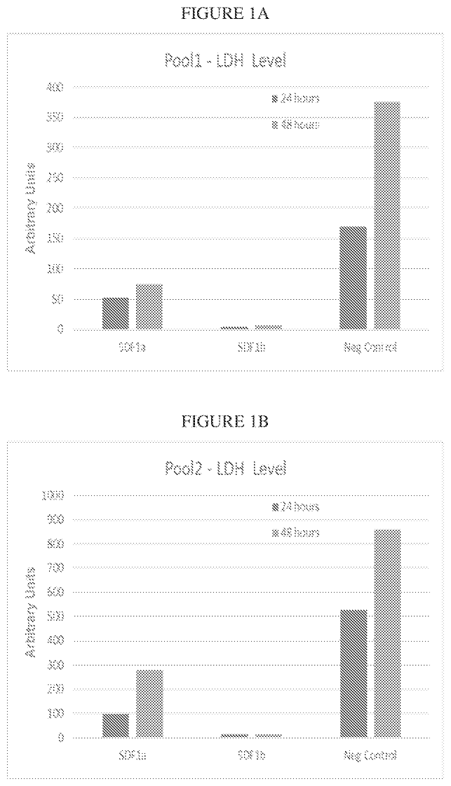

Data (background subtracted) from a representative experiment are provided in Table 1 and FIG. 1A. Data from a second representative experiment are provided in FIG. 1B.

TABLE 1
LDH and CXCL12 Levels

| Cytokine | LDH-24 hr | LDH-48 hr | Cytokine Conc. |
|---|---|---|---|
| Control | 170 | 375 | N/A |
| SDF1a | 52 | 75 | ~100 nM |
| SDF1b | 4 | 7 | ~400 nM |

These data indicate that the expression of CXCL12 by beta islet cells protects the beta islet cells from immune cell attack. Beta cells expressing SDF1b/CXCL12b, which was expressed at a higher level than SDF1a/CXCL12a in this experiment, show essentially no cytotoxicity in the presence of PBMCs. These data also show the importance of administering exogenous cytokines during implantation of the transgenic beta cells, since newly-implanted cells would be similar to the control cells in that they would have little or no fugetactic agent in the vicinity of the cells for at least a period of time after implantation, and therefore would be vulnerable to attack by host immune cells.

### Example 3: Alternative Preparation of Transgenic Beta Cells

Beta cells are isolated from a subject having type 1 diabetes are transfected or infected in vitro with a retroviral expression vector encoding CXCL12 or a control retroviral vector that does not encode CXCL12. Transgenic beta cells harboring the retroviral vector encoding CXCL12 are assayed for expression of fugetactic amounts of CXCL12 using a boyden chamber assay as previously described in Poznansky et al., Journal of Clinical Investigation, 109, 1101 (2002).

### Example 4: Effect of Forced Senescence of Transgenic Beta Cells on Transgenic Cytokine Expression

Beta cells were prepared as described in Example 1. Expression levels of SDF1a/CXCL12a and SDF1b/CXCL12b were assayed by ELISA before Mitomycin C treatment to determine baseline expression (“Before”). Medium was replaced with fresh medium containing 10 ug/mL Mitomycin C—an agent known to induce senescence. Cells were returned to the incubator for 2 hours. The mitomycin C containing medium was removed by gentle pipetting. The cells were washed with PBS twice. After the second wash, the cells were fed fresh complete medium. SDF1a/CXCL12a or SDF1b/CXCL12b expression was determined by ELISA assay.

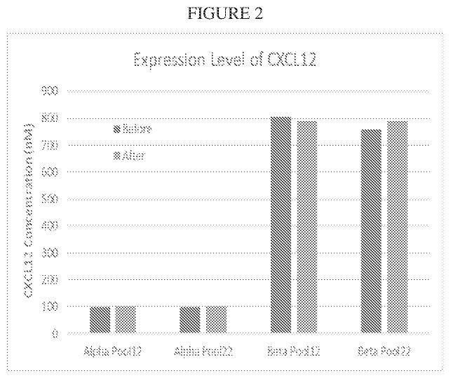

Data from two representative experiments are shown in FIG. 2A. SDF1a/CXCL12a and SDF1b/CXCL12b expression is not affected by forced senescence of the transgenic beta cells.

### Example 5: Effect of Forced Senescence of Transgenic Beta Cells on PBMC Challenge

Beta cells were prepared as described in Example 1. Cells were treated with Mitomycin C or control as described in Example 4. Cells were contacted with PBMCs as described in Example 2.

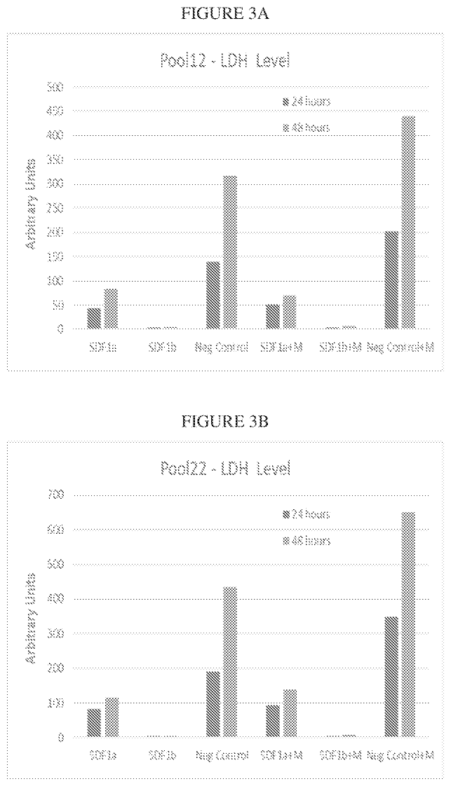

Data from two representative experiments are shown in FIGS. 3A and 3B. LDH levels are not affected by forced senescence of the transgenic beta cells.

### Example 6: Effect of Forced Senescence of Transgenic Beta Cells on Insulin Production

Beta cells were prepared as described in Example 1. Cells were treated with Mitomycin C or control as described in Example 4.

Full growth medium was replaced with 1 mL of Medium 2 and maintained on Medium 2 for two days with medium replacement every 24 hours. On day 3, the beta cells were challenged with the hyperglycemic medium (4.5 g/L glucose, with reduced supplement addition). Samples of conditioned media were taken 24 hours following hyperglycemic challenge, and insulin expression was measured by sandwich ELISA.

The results evidence that transgenic beta cells and transgenic senescent beta cells produced substantially equal amounts of insulin as the control beta cells in response to glucose.

### Example 7: In Vivo Evaluation of Transgenic Beta Cells

Humanized mice having a humanized immune system, see, e.g., N. Walsh, “Humanized mouse models of clinical disease,” Annu Rev Pathol 2017, 12, 187-215; E. Yoshihara et al., are administered either the transgenic beta cells expressing fugetactic amounts of CXCL12, or the control transgenic beta cells and the production of insulin and survival of the transgenic beta cells in the mice are assayed at various time points after the initial administration. It is contemplated that the transgenic beta cells expressing fugetactic amounts of CXCL12 will survive for longer periods than the control transgenic beta cells. It is also contemplated that the insulin levels in mice receiving the transgenic beta cells expressing fugetactic amounts of CXCL12 will also have higher amounts of insulin than mice receiving the control transgenic beta cells and the higher levels of insulin will persist for longer periods of time as compared to the levels in mice administered the control transgenic beta cells.

Humanized mice having a humanized immune system, see e.g., N. Walsh, “Humanized mouse models of clinical disease,” Annu Rev Pathol 2017, 12, 187-215; E. Yoshihara et al., are administered either genetically modified beta cells overexpressing CXCL12 from an endogenous CXCL12 gene, or control beta cells and the production of insulin and survival of the beta cells in the mice are assayed at various time points after the initial administration. It is contemplated that the genetically modified beta cells overexpressing CXCL12 will survive for longer periods than the control beta cells that were not genetically modified to overexpress CXCL12. It is also contemplated that the insulin levels in mice receiving the genetically modified beta cells overexpressing CXCL12 will also have higher amounts of insulin than mice receiving the control beta cells and the higher levels of insulin will persist for longer periods of time as compared to the levels in mice administered the control beta cells.

Optionally, the cells are treated with an agent that cross-links the DNA within the cell to prevent cell division (e.g., mitomycin C).

The invention is further illustrated by the following proposed examples.

A male mouse is treated with streptozotocin to induce diabetes. The mouse is then implanted into the peritoneal cavity with a degrading hydrogel containing luciferase, wherein the degradation half-life of the hydrogel is about 4 days. Subsequently, genetically modified murine beta cells altered to express murine CXCL12 are injected into the hydrogel by needle injection using the bioluminescence of luciferase to guide injection. The cells are monitored to ensure their survival. In one embodiment, such is evidenced by control of the blood sugar in the mouse starting at day 10.

A male patient newly diagnosed with T1D is implanted into the peritoneal cavity with a degrading hydrogel containing luciferase, wherein the degradation half-life of the hydrogel is about 4 days. Subsequently, genetically modified human beta cells altered to express a fugetactic amount of human CXCL12 beta (as described in U.S. Pat. No. 10,696,950) are injected into the hydrogel by needle injection using the bioluminescence of luciferase to guide injection. The cells are monitored to ensure their survival. In one embodiment, such is evidenced by control of the blood sugar in the patient starting at day 10.

The foregoing description has been set forth merely to illustrate the invention and is not meant to be limiting. Since modifications of the described embodiments incorporating the spirit and the substance of the invention may occur to persons skilled in the art, the invention should be construed broadly to include all variations within the scope of the claims and equivalents thereof.

1. A method for protecting genetically engineered, mammalian cells that express an effective concentration of a fugetactic agent that protects said cells against immune cell attack after implantation into a subject said method comprises:
a) selecting a cell or a population of mammalian cells that are genetically engineered to express an effective concentration of a fugetactic agent in the microenvironment;
b) implanting into a subject a rapidly biodegradable matrix or gel that is impregnated with and elutes an effective amount of the same fugetactic agent into the microenvironment surrounding the matrix or gel; and
c) implanting with or into said matrix or gel said cell or a population of said cells whereby said cells are protected from graft versus host immune attack after implantation, wherein said rapidly degrading biodegradable matrix or gel has a degradation period of about 10 days or less.

2. The method of claim 1, wherein said implanted cells are selected from the group consisting of genetically engineered allogeneic cells and genetically engineered xenogeneic mammalian cells.

3. The method of claim 1, wherein the matrix or gel comprises one or more components selected from the group consisting of: oxygen releasing agents, glucose, an anti-inflammatory agent, and an antibiotic.

4. The method of claim 1, wherein the matrix or gel is a hydrogel containing sufficient water to avoid inducing osmotic shock at the site of implantation.

5. The method of claim 4, wherein the hydrogel is selected from collagen/gelatin, chitosan, hyaluronic acid, chondroitin sulfate, alginate, agar/agarose, fibrin, polyethylene glycol, polyethylene oxide, polylactide, and polyvinyl alcohol, provided that the hydrogel has been a rapid degradation period in vivo.

6. The method of claim 1, wherein the fugetactic agent is selected from the group consisting of: a CXCL12 isoform, gp120, CXCR4 ligands, IL-8, CXCR4-binding antibodies, and CXCL13.

7. The method of claim 6, wherein the fugetactic agent is a CXCL12 isoform.

8. The method of claim 7, wherein the CXCL12 isoform is a CXCL12 alpha or a CXCL12 beta.

9. The method of claim 8, wherein the CXCL12 isoform is a CXCL12 beta.

10. A biologically compatible, biodegradable, implantable matrix or gel comprising an effective amount of a fugetactic agent and genetically engineered, immune protected, mammalian cells, wherein said mammalian cells express an effective amount of a fugetactic agent so as to resist destruction by human immune cells wherein said matrix or gel biodegrades in vivo within 10 days of implantation.

11. The biologically compatible, implantable matrix or gel of claim 10, wherein said fugetactic agent is selected from the group consisting of: a CXCL12 isoform, gp120, a CXCR4 ligand, an interleukin-8 (IL-8), a CXCR4-binding antibody, and a CXCL13.

12. The biologically compatible, implantable matrix or gel of claim 11, wherein the fugetactic agent is a CXCL12 isoform.

13. The biologically compatible, implantable matrix or gel of claim 12, wherein the CXCL12 isoform is a CXCL12 alpha or a CXCL12 beta.

14. The biologically compatible, implantable matrix or gel of claim 13, wherein the CXCL12 isoform is a CXCL12 beta.

15. The biologically compatible, implantable matrix or gel of claim 10, wherein said implanted mammalian cells are genetically engineered allogeneic human beta cells.

16. The biologically compatible, implantable matrix or gel of claim 10, wherein the matrix or gel comprises one or more components selected from the group consisting of: oxygen releasing agents, glucose, an anti-inflammatory agent, and an antibiotic.

17. The biologically compatible, implantable matrix or gel of claim 10, wherein the matrix or gel is a hydrogel containing sufficient water to avoid inducing osmotic shock at the site of implantation.

18. The biologically compatible, implantable matrix or gel of claim 17, wherein the hydrogel is selected from the group consisting of: a collagen/gelatin, a chitosan, a hyaluronic acid, chondroitin sulfate, an alginate, an agar/agarose, a fibrin, a polyethylene glycol, a polyethylene oxide, a polylactide, and a polyvinyl alcohol, provided that the hydrogel is rapidly degrading in vivo.

19. The biologically compatible, implantable matrix or gel of claim 18, wherein the matrix or gel is a hydrogel containing sufficient water to avoid inducing osmotic shock.

20. The method of claim 2, wherein the genetically engineered allogeneic cells are genetically engineered allogeneic beta cells.


SEQUENCE LISTING (10 sequences)

SEQ ID NO: 1 - protein, 89 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNK

SEQ ID NO: 2 - protein, 93 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKRFKM

SEQ ID NO: 3 - protein, 119 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKGRREEKVGKKEKIGKKKRQKKRKAAQKRKN

SEQ ID NO: 4 - protein, 140 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNNLISAAPAGKRVIAGARALHPSPPRACPTARALCEIRLWPPPEWSWPSPGDV

SEQ ID NO: 5 - protein, 90 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNNC

SEQ ID NO: 6 - protein, 100 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKIWLYGNAETSR

SEQ ID NO: 7 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 8 - DNA, 375 bp, Homo sapiens
atgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagaggttcaagatgacgcgtacgcggccgctcgagcagaaactcatctcagaagaggatctggcagcaaatgatatcctggattacaaggatgacgacgataaggtttaa

SEQ ID NO: 9 - DNA, 363 bp, Homo sapiens
atgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagacgcgtacgcggccgctcgagcagaaactcatctcagaagaggatctggcagcaaatgatatcctggattacaaggatgacgacgataaggtttaa

SEQ ID NO: 10 - protein, 8 aa, Artificial Sequence
  Description of Artificial Sequence Synthetic peptide
DYKDDDDK